(19) United States
(12) Patent Application Publication
(10) Pub. No.: US 2004/0219540 A1
(43) Pub. Date: Nov. 4, 2004

(54) Method for detecting pancreatic and gastro-intestinal illnesses

(76) Inventor: Manfred Nilius, Magdeburg (DE)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/466,061

(22) PCT Filed: Dec. 11, 2001
(86) PCT No.: PCT/EP01/14511

(30) Foreign Application Priority Data
Jan. 17, 2001 (DE) 101 01 792.8

(51) Int. Cl. C12Q 1/68; G01N 33/53; C12N 15/00; C07H 21/04

(52) U.S. Cl. 435/6; 435/69.1; 435/320.1; 435/325; 530/350; 536/23.5

(57) ABSTRACT

The present invention relates to a method for the detection of gastric and pancreatic diseases using antibodies in serum and feces.

### FIELD OF THE INVENTION

The present invention relates to methods for the detection of pancreatic and gastrointestinal diseases, particularly to methods for the detection of pancreatic carcinomas, chronic pancreatitis, gastric lesions, and inflammatory intestinal diseases.

### BACKGROUND OF THE INVENTION

Pancreatic diseases, such as pancreatic carcinoma or chronic pancreatitis, just as much as gastrointestinal diseases, for example, gastric lesions or inflammatory intestinal diseases, represent serious threats to the human and animal body. An early and specific as possible a diagnosis of such diseases is indispensable for a therapy promising success. Heretofore, nearly all diseases of the gastrointestinal tract have been diagnosed with invasive methods (endoscopy, colonoscopy, ERCP, etc.) with tissue sampling, supported by imaging methods (sonography, X-rays, computer tomography, etc.) as standard methods. In the initial phase of these investigations, a few laboratory methods have become established as routine methods which can provide information about an insufficiency of certain internal organs, such as the pancreas, for example. These laboratory methods, however, are mostly expensive and often have to be performed on fresh material (gastric juice, pancreatic secretion, duodenal secretion, etc.) within a short time, since a detection of specific factors is often no longer attained due to the enzymatic composition of the sample material (this is so above all for enzyme-substrate reactions). If certain parameters in the patient's serum are determined, these values are often not reliable to the desired degree (large intra- and inter-individual fluctuations) and exhibit extremely low sensitivity.

In a few diseases, (chronic pancreatitis, gastric infection with Helicobacter pylori, bacterial colonization of the intestine, etc.) it has been possible to establish respiratory tests (for example, ¹³C-urea respiratory test, H₂-glucose respiratory test, ¹³C-triglyceride respiratory test, etc.), which however can only be performed up to now at a few centers, since corresponding expensive equipment (for example, a mass spectrometer) has to be available, and the costs, conditioned by expensive ¹³C labeling, cannot be further reduced.

These methods are therefore also usually unsuitable for corresponding preventive screening programs.

There are up to now only a few diagnostic tests with which given diseases can be detected in the patient's feces. For example, there is the occult blood test for the detection of occult blood in the feces as a screening method for intestinal carcinoma, the detection of pancreatic elastase in the feces for the diagnosis of chronic pancreatitis, and the recently developed Helicobacter pylori feces test for detection of Helicobacter pylori infection in the stomach. In the last two tests, the detection if performed by antibody technology.

In relation to the test for pancreatic elastase, it can be determined that the test is admittedly a usable method for diagnosis of severe exocrine pancreatic insufficiency, but however detects mild to moderate functional limitation relatively poorly.

A reliable method for non-invasive differentiation of suspected malignant pancreatic changes is still not available. As candidates for non-invasive diagnosis of pancreatic carcinoma, markers which detect given gene mutations (K-Ras, p16, p53) are above all the subject of discussion. Since the individual genes come into action differently in the course of the emergence of a carcinoma over time, they are also associated with different probability with the emergence of a carcinoma.

Thus, for example, K-Ras mutations are already detected in a premalignant stage, that is, in a stage of the “emergence” of a carcinoma, while the tumor suppressor genes p16 and p53 are inactivated when a carcinoma is present. Genes which participate early in the emergence of the carcinoma have the disadvantage, though, that they can also be detected in preliminary stages of tumor emergence, so-called “pre-malignant” states, and in patients with chronic pancreatitis without tumors, which in turn limits their predictive power. A combination of different tumor markers among these, or of other specific markers for pancreatic carcinoma, is therefore to be sought.

The same holds for the detection of CA 19-9 used as a course parameter, a tumor marker which is determined in patient serum as a course marker and recurrence indicator. Studies show that as many as 5/7 patients with a chronic pancreatitis have elevated CA 19-9 serum levels, and after a successful operation of a pancreatic carcinoma a normalization of the serum CA 19-9 level occurs in only 10-29% of cases.

Up to now, a commercial kit for detection of K-Ras by means of PCR technique is available (Elucigene K-Ras7; Zeneca Diagnostics), but this technique is time-consuming and expensive and requires, as do all molecular biological methods, a very careful (sterile) environment for the test material and for the performance of the test method, in order to prevent DNA contamination from the surroundings, which can give a false positive or false negative detection.

The detection of carboxypeptidase A (CPA) presents itself as a further marker for the diagnosis of pancreatic carcinoma. This enzyme is exclusively formed in the pancreas as a precursor (“proenzyme”), procarboxypeptidase A (PCPA). The enzyme is converted into active carboxypeptidase by splitting the propeptide.

Elevated PCPA is found in the serum of pancreatic tumor patients. Furthermore, carboxypeptidase A is secreted by the healthy pancreas in only small amounts (about 5% of the enzyme secretion) in comparison with other pancreatic enzymes (elastase, amylase, etc.). It is therefore to be expected that a pathological change in the pancreas (pancreatitis, pancreatic carcinoma) will lead to a fall of the enzyme below the detection limit or a rise of the “unripe” proenzyme (PCPA). A further advantage is that carboxypeptidase A is undamaged when passing through the intestine and may be quantified in the feces.

Up to now there is no commercial detection kit for the determination of CPA or PCPA in body fluids or feces.

In sum, it can be stated that a reliable, non-invasive diagnosis of chronic changes in most gastrointestinal diseases is still not available.

In chronic inflammatory intestinal diseases such as Crohn's disease or ulcerative colitis, in general a very long time passes between the appearance of the first symptoms and reaching a diagnosis, and in many cases this makes the carrying out of a curative treatment much more difficult or even impossible. Crohn's disease and ulcerative colitis are chronic inflammatory intestinal diseases of unclear etiology and for which there are at present neither satisfactory diagnostic nor therapeutic possibilities available. The methods at present used for the diagnosis of Crohn's disease or ulcerative colitis include establishing inflammation parameters and microbiological and serological investigations of body fluids and feces. In addition, sonographic and endoscopic investigation methods are used in a few cases. Moreover, methods using radioactive isotopes such as ¹¹¹indium or ⁹⁹technetium were developed, in the context of which leucocytes were labeled with these isotopes and detected. Although these methods have high sensitivity and specificity for the detection of Crohn's disease and ulcerative colitis, they are found to be technically complicated, expensive, and a burden on the patient. These diagnostic methods cannot be used for certain patient groups such as children or pregnant women.

Gentler methods were therefore developed which already make possible an early detection of specific disease pictures on the basis of antibody tests. Thus, for example, U.S. Pat. No. 5,455,160 describes methods for the diagnosis of oncological diseases of the gastrointestinal tract and also of Crohn's disease and ulcerative colitis. The publication discloses the use of antibodies against calprotectin, particularly the use of an enzyme immunological test for the quantification of calprotectin in patients' feces, an elevated amount of calprotectin giving an indication of one of the said diseases.

DE 41 07 765 A1 describes a method for the recovery of a highly specific pancreatic elastase 1-antibody which reacts both with body fluids and with feces. An antibody recovered by this method is suitable for the diagnosis of chronic and acute pancreatitis in feces.

SLPI (Secretory Leucocyte Proteinase Inhibitor), a protein of 11.7 kDa size with protease-inhibiting function, is known from Si-Tahar (Gastroenterology 118 (2000), 1061-1071) as a possible therapeutic agent against intestinally damaging influences such as microbial infections.

From WO 94/06454, the use of SLPI as a prophylactic agent against an HIV infection is known. In WO 99/17800, the use of SLPI in pulverized form as a pharmaceutical agent is described. The pulverized offering form especially described there is for the use of SLPI in an inhalation therapy. In U.S. Pat. No. 5,633,227, the use of SLPI is set forth for the treatment of asthma or allergic rhinitis. JP 07103977 sets forth immunological sandwich tests for SLPI or SLPI-elastase complexes, SLPI fragments with the amino acid residues 1-54 and 55-107 being known. It is described that by means of the described system, diseases of the respiratory tract can be detected. From JP 03279862, antibody-based test methods for SLPI are likewise known.

For SLPI it is known that this protein has inhibitory activity against chymotrypsin-type proteases (such as pancreatic elastase) and trypsin-type proteases. It is known for the former activity that this is associated with the C-terminal region, and for the latter activity, with the N-terminal region of SLPI. U.S. Pat. No. 5,851,983 describes shortened and fusion proteins produced based on these discoveries, and also pharmaceutical application possibilities of the same. Finally, WO 96/08275 discloses further fragments of SLPI and their use for inhibiting tryptases.

### BRIEF SUMMARY OF THE INVENTION

The technical problem on which the present invention is based is thus to provide a detection method for pancreatic and gastrointestinal diseases, particularly pancreatic carcinoma, chronic pancreatitis, gastric lesions and inflammatory intestinal diseases, which is simple to perform, gentle, cost-effective, and also rapid in execution.

The invention solves this problem by the provision of a method for the detection of a disease of the human or animal body, selected from the group consisting of pancreatic carcinoma, chronic pancreatitis, gastric lesions, and inflammatory intestinal disease, wherein SLPI (Secretory Leucocyte Protease Inhibitor), fragments or complexes thereof, recovered from serum or feces of the human or animal body, are detected, in particular wherein the concentration of SLPI, fragments of complexes thereof are determined in serum or feces. According to the invention, it could surprisingly be shown that with the use of immunological means, particularly of monoclonal or polyclonal antibodies, a detection of the said diseases in serum or feces of the human or animal body is possible, in that the presence or absence of SLPI, complexes or fragments thereof, particularly the concentration of SLPI present, is determined in comparison with healthy control persons.

### BRIEF DESCRIPTION OF THE DRAWINGS

The invention is described in detail using the following examples and the accompanying Figures.

FIG. 1 shows a histogram of SLPI concentration in feces,

FIG. 2 shows a histogram of elastase-1 concentration in feces; and

FIGS. 3 and 4 respectively show a histogram of SLPI concentration in serum.

### DETAILED DESCRIPTION OF THE INVENTION

Patients with chronic pancreatitis or pancreatic carcinoma can be diagnosed according to the invention in that SPLI appears in their feces, in particular in high concentrations compared with healthy persons. The feces of healthy human or animal bodies has very little or no SLPI. According to the invention, not only can chronic pancreatitis or pancreatic carcinoma be determined by means of the present invention in the feces of patients, but moreover the degree of detection can be quantified by means of a SLPI concentration determination. It could be shown that the SLPI concentrations in the feces correlate with the concentrations of elastase-1 present there. It could in particular be shown that in patients with chronic pancreatitis, the SLPI concentrations in the feces are correlated with the elastase-1 concentrations, and that also the SLPI concentrations in healthy control persons are correlated with the elastase-1 concentration of these persons. The same holds for the SLPI concentration of patients with pancreatic carcinoma, likewise correlated with the elastase-1 concentrations of these patients.

The invention is surprising because, among other things, SLPI in the intestinal tract could be shown not to be immediately decomposed in each case. Rather, in patients with chronic pancreatitis and pancreatic carcinoma, SLPI is detected in the feces.

The invention thus relates to methods for the immunological detection of pancreatic diseases by the detection of SLPI in the feces of human or animal bodies.

It could moreover be shown that in healthy control persons who have no gastric lesions such as gastritis or ulcer, the SLPI concentration in serum is lower than in patients with such lesions. According to the invention, the present method of immunological detection of pancreatic and gastrointestinal diseases thus also serves for the diagnosis of gastric lesions, elevated SLPI concentrations being detected as against normal, healthy comparison persons. The invention therefore relates in particular to methods for the immunological detection of gastrointestinal diseases by the detection of SLPI in the serum of human or animal bodies.

In connection with the present invention, the concept “immunological means” is to be understood to mean in particular the use of antibodies for the detection of antigens, in particularly of SLPI, SLPI-containing complexes, for example, protein-protein complexes of homopolymeric or heteropolymeric composition, or SLPI fragments. In connection with the present invention, the concept SLPI hereinafter is to be understood to also mean its complexes with other proteins, such as a SLPI-elastase-1 complex, or fragments of SLPI, for example C- or N-terminal fragments. Such fragments can for example have amino acid residues 1-54 (N-terminal fragment) or 55-107 (C-terminal fragment). As regards the said position data, reference is made to U.S. Pat. No. 5,851,983 and the numbering given there in SEQ ID No. 4. The disclosure content of U.S. Pat. No. 5,851,983 and also of WO 96/08275 as regards the fragments of SLPI disclosed there is incorporated herein by reference and makes clear that protection is claimed for the fragments named there in combination with the present method according to the invention.

In connection with the present invention, the concept antibody is to be understood to include both monoclonal and polyclonal antibodies and fragments thereof, so long as these specifically detect and bind to SLPI, SLPI-containing complexes, for example, SLPI-elastase-1 complexes, or SLPI fragments. In a preferred embodiment, the antibodies or fragments thereof bind to no other antigens. The antibodies or fragments can of course be modified, for example, conjugated, associated, or covalently or non-covalently bonded, with other molecules or portions thereof, for example with color labels, radioactive labels, enzymes releasing measurable reactions, such as phosphatases, peroxidases, enzyme substrates, fluorescing substances, chemiluminescent substances, cytotoxic agents, spacers, support materials or the like. The labeled, conjugated or non-modified antibodies can be present in soluble or immobilized form, for example on support matrices or small spheres. For example, the enzyme-labeled antibodies can be used in a second enzyme amplification system.

According to the invention, in a preferred embodiment there is understood by the use of immunological means, the bringing into contact of a sample, that is, of serum or feces, in particular homogenized feces, with an immunological agent, including at least one antibody which specifically detects the antigen, thus SLPI.

The invention therefore also relates to the use of antibodies against SLPI for the qualitative and/or quantitative determination of SLPI in feces or serum of a human or animal body. According to the invention it is thus possible by the use of antibodies to SLPI to detect gastrointestinal diseases specifically in serum and pancreatic carcinoma or chronic pancreatitis in feces.

The methods according to the invention with the use of immunological means can preferably be embodied as sandwich ELISA, indirect or competitive ELISA, and the use of HTP (high throughput) methods is particularly preferred.

In the context of the sandwich ELISA according to the invention, the provision of at least two different monoclonal antibodies is necessary, which preferably are directed against different epitopes, alternatively however even the same epitope, of SLPI. The at least two antibodies can for example be present, soluble and/or supported, in a homogeneous or heterogeneous system.

According to the invention it is also of course also possible to embody the immunological means as a system of three different antibodies, one of the antibodies being present in a heterogeneous phase and the two other antibodies being soluble. One of the two soluble antibodies is labeled, while the other is unlabeled. The soluble antibody is in this embodiment directed against the unlabeled antibody.

Methods according to the invention carried out with the use of immunological means thus require, in a particularly preferred embodiment, the incubation of the serum or, preferably homogenized and if necessary diluted, feces with at least two different antibodies, which are specific for SLPI. In a preferred embodiment, one of the two antibodies is bound to a solid phase in the usual manner. The further antibody is advantageously present in soluble form and bears a label in a preferred embodiment. If according to the invention further antibodies are used, in a preferred embodiment only one of these bears this one label.

It is provided in an embodiment according to the invention that either an antibody which can unspecifically bind with SLPI or, particularly preferred, an antibody which can specifically bind to SLPI, is bound to a solid phase. This antibody bound to the solid phase is then incubated with the serum or feces and, if necessary after a washing step, incubated with a second antibody which can specifically bind to SLPI, is present in a soluble form, and bears a label. Provided that the antibody bound to the solid phase is an antibody which can unspecifically bind to SLPI, not only SLPI but also other antigens bind to the solid phase. The second antibody, which however binds specifically to SLPI, now reacts specifically only with SLPI, or respectively with the complex of SLPI with the first antibody, so that only SLPI molecules specifically bear a labeled antibody and thus can be quantified after separation of the solid from the liquid phase.

According to the invention, it can thus also be provided, for example, in the context of a sandwich ELISA, to bind an unlabeled SLPI-specific first antibody for example to a carrier, and to add the test solution or suspension, the SLPI present in the test solution or suspension being bound by the first antibody. A second labeled antibody against SLPI is then added, which specifically reacts with SLPI or the complex of SLPI and first antibody. By means of the labeling of the second antibody, the amount of SLPI can be determined by the use of a standard solution.

If a first antibody binding specifically to SLPI is fixed to the solid phase, then only SLPI will specifically also be bound to the solid phase. In the incubation with the second, soluble antibody, this also reacts exclusively with SLPI. After separation of the solid from the liquid phase, an exact quantification of SLPI can be performed by means of the labeling of the second antibody.

In a particularly preferred embodiment, the invention also provides that a microtiter plate is coated with a first antibody specifically or unspecifically binding to SLPI, that the serum or feces is brought into contact with the coated microtiter plate and, after washing of unbound constituents, a labeled, for example biotin labeled, second antibody against SLPI is added to the microtiter plate, the microtiter plate being incubated under conditions such that the second antibody can bind. The solid phase is then separated from the liquid phase and either the label is directly detected or, with the use of an enzyme-labeled second antibody, a substrate is added and the conversion of the substrate is quantitatively determined. Thus it can be provided that the biotin-labeled second antibody reacts in a second incubation with a conjugate of peroxidase (POD) and streptavidin. The peroxidase is able to oxidize the substrate ABTS (2, 2′-azino-bis-(3-ethylbenzothiazoline-6-sulfonic acid) diammonium salt. Oxidized ABTS can then be photometrically determined.

In a particularly preferred embodiment, the first antibody is bound to a support matrix, for example, a nonwoven, woven, or membrane structure, so that does not as usual represent the floor of the depression of an ELISA immunoplate, but is directly bound into the matrix. Preferred matrices are hollow fiber membranes or microporous flat membranes, which in an advantageous embodiment of the invention can also be provided with ion exchange groups.

Polyclonal antibodies can be produced in that animals are immunized with isolated and completely purified SLPI or fragments thereof, and antisera with polyclonal antibodies are thus obtained in a manner known per se. Monoclonal antibodies are produced by methods known per se. The antibodies against SLPI used according to the invention can be commercially obtainable antibodies.

The gastric lesions which can be detected according to the invention can be symptoms of a gastritis or an ulcer. The inflammatory diseases which can be detected according to the invention can for example be attributed to a Helicobacter pylori infection or symptoms of Crohn's disease or ulcerative colitis.

In a particularly preferred embodiment of the present invention, it is provided that the elastase-1 concentration is determined simultaneously with the detection of SLPI in the patient's serum or feces. Human pancreatic elastase withstands passage through the intestine undamaged, and can be quantified as protein in the feces. The elastase-1 concentration in the feces mirrors the exocrine pancreatic function. Values greater than 200 μg elastase per gram indicate a normal exocrine pancreatic function. Values smaller than 200 μg elastase per gram of feces indicate an exocrine pancreatic insufficiency. In an acute pancreatitis, elastase-1 can be released into the serum in the acute inflammatory phase, so that quantifying the elastase in serum permits the diagnosis or exclusion of this disease. A high concentration of elastase-1 in serum thus points to a pancreatitis.

Further advantageous embodiments of the invention will become apparent from the dependent claims.

### EXAMPLE 1

### Detection of SLPI in Feces of Patients with Chronic Pancreatitis

From 42 patients without chronic pancreatitis, 6 patients with pancreatic carcinoma, and 19 patients with chronic pancreatitis respectively 100 mg of feces were recovered, weighed, and extracted with extraction buffer (phosphate-buffered sodium chloride solution, pH 7.2, with detergent and sodium azide) and diluted.

The extraction was performed by repeatedly strongly mixing the feces suspension at room temperature using a test tube shaker. The feces must be well homogenized in order to ensure a complete extraction. After extraction for at least five minutes, mixing is finally performed once more. After settling of the particles, the feces sample extracts are diluted as follows.

The dilution of the sample extracts took place for measurement of SLPI at a dilution of 1:100 and for comparative measurement of elastase-1 at a dilution of 1:500.

The SLPI concentration in feces was determined by means of a SLPI ELISA (Quantikine®, Cat. No. DP 100, 6/99) of the R&D Systems company (R&D Systems GmbH, Wiesbaden, Germany).

This ELISA uses a polystyrene microtiter plate having 96 wells, coated with monoclonal mouse antibody against human SLPI. After pipetting the extract into the wells, SLPI is bound by the immobilized monoclonal antibody. After washing off unbound substances, an enzyme-linked polyclonal antibody which is specific for human SLPI is added to the wells (polyclonal antibody against human SLPI, bound to horseradish peroxidase). After washing off unbound antibody-enzyme reagent, a substrate solution is added to the wells and the color development is measured as a measure of bound SLPI.

The concentration of pancreatic elastase-1 was determined by means of an elastase-1 ELISA of the ScheBo-Tech® company (Giessen, Germany, Cat. No. 07, April 2000, EP 0 547 059 B1).

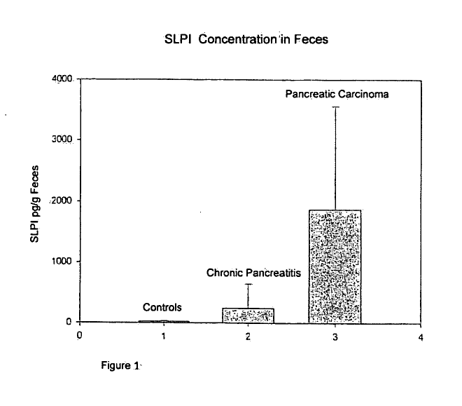

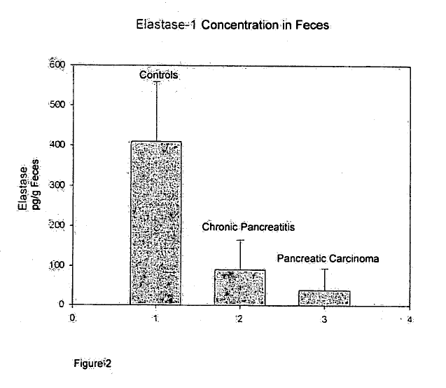

The results shown in FIGS. 1 and 2 below were obtained:

FIG. 1 shows the SLPI concentration in feces of patients (SLPI in picograms per gram feces), shown for the patient group without chronic pancreatitis, the patient group with pancreatic carcinoma, and the patient group with chronic pancreatitis. It shows clearly that the members of the control group have no or very little SLPI in feces, while the two patient groups with chronic pancreatitis and pancreatic carcinoma have considerably elevated SLPI concentrations in feces.

FIG. 2 shows the concentration measurements of elastase-1 performed on the same patient groups. The healthy control group has an elastase content clearly lying above 200 μg/g feces, while the two patient groups with chronic pancreatitis and pancreatic carcinoma have significantly reduced elastase-I concentrations in feces.

### EXAMPLE 2

### EXAMPLE 2a

### Detection of SLPI in Serum of Patients with or without Gastric Disease

Of 17 patients, with normal stomach findings (negative) and 33 patients with a gastric or duodenal ulcer (ulcus ventriculi or ulcus duodeni), respectively 5 ml of blood was recovered and serum was obtained by centrifuging at 3,000 rpm at room temperature for 10 minutes.

The dilution of the serum samples was performed for the measurement of SLPI at a dilution of 1:100.

The SLPI concentrations in serum were determined by means of SLPI ELISA (Quantikine®, Cat., No. DP 100, 6/99) of the R&D Systems company (R&D Systems GmbH, Wiesbaden, Germany). The ELISA was performed as described in Example 1.

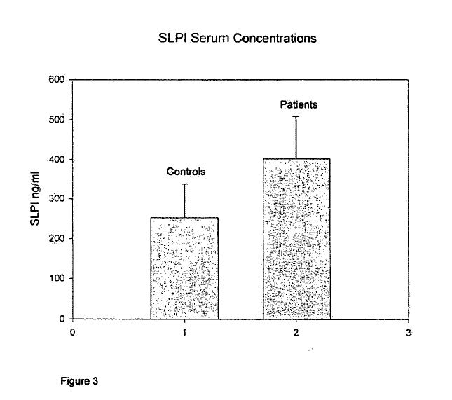

FIG. 3 shows the SLPI concentration in ng/ml in the control group and the patients.

### Results

In comparison with the control group, the patient group showed significantly higher serum SLPI concentrations.

### EXAMPLE 2b

Respectively 5 ml of blood was recovered from 17 patients with normal stomach findings(controls), 16 patients with chronic gastritis, and 18 patients with ulcers, and serum was obtained by centrifuging at 3,000 rpm at room temperature for 10 minutes.

The SLPI concentrations in serum were determined by means of SLPI ELISA (Quantikine®, Cat., No. DP 100, 6/99) of the R&D Systems company (R&D Systems GmbH, Wiesbaden, Germany).

The ELISA was performed as described in Example 1.

Both patients with chronic gastritis and patients with ulcers (ulcus ventriculi or ulcus duodeni) had a significantly elevated SLPI concentration in serum.

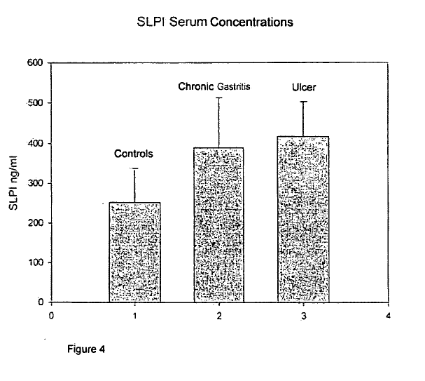

FIG. 4 shows the SLPI concentrations in ng./ml in patients with normal stomach findings, patients with chronic gastritis, and patients with ulcers (ulcus). The Figure shows clearly that the healthy control group has a considerably lower serum SLPI concentration than both the patient group with chronic gastritis and that with ulcers.

### Results

SLPI concentrations are also suitable as markers for gastrointestinal lesions.

1. Method for the detection of a disease of the human or animal body, selected from the group consisting of pancreatic carcinoma, chronic pancreatitis, gastric lesions, and inflammatory intestinal disease, wherein serum or feces of the body is recovered and SLPI (Secretory Leucocyte Protease Inhibitor), a fragment or a complex thereof, present therein is detected by bringing into contact with immunological means.

2. Method according to claim 1, wherein the feces is homogenized before being brought into contact with the immunological means.

3. Method according to claim 1, wherein the immunological means are antibodies, particularly labeled antibodies, against SLPI or fragments or complexes thereof.

4. Method according to claim 3, wherein the SLPI-antibody reaction is detected by means of an ELISA test.

5. Method according to claim 1, wherein the gastric lesion is to be attributed to gastritis or to an ulcer.

6. Method according to claim 1, wherein the inflammatory intestinal disease is Crohn's disease or ulcerative colitis.

7. Method according to claim 1, wherein, in serum or feces of the body, the elastase-1 concentration is also detected as well as the SLPI-antibody reaction.

8. Method according to claim 1, wherein the antibodies are monoclonal antibodies.

9. Method according to claim 1, wherein the complex is a SLPI-elastase-1 complex.

10. Method according to claim 1, wherein the fragment of SLPI is the N-terminal region of SLPI with the amino acid residues 1-54.

11. Method according to claim 1, wherein the fragment of SLPI is the C-terminal portion of SLPI with the amino acid residues 55-107.

12. Use of antibodies directed against SLPI fragments or complexes thereof for the detection of a disease of a human or animal body in feces or serum of the human or animal body, selected from the group consisting of pancreatic carcinoma, chronic pancreatitis, gastric lesion, and inflammatory intestinal disease.
